(19) United States
(12) Patent Application Publication
(10) Pub. No.: US 2024/0175736 A1
(43) Pub. Date: May 30, 2024

(54) FLOW COMPUTER WITH INTERFACE TO AN EXTERNAL API FOR DATA

(71) Applicant: Schneider Electric Systems USA, Inc., Foxboro, MA (US)

(72) Inventors: James Redmond, Richmond (CA); Zackery Sobin, Raleigh, NC (US)

(73) Assignee: Schneider Electric Systems USA, Inc., Foxboro, MA (US)

(21) Appl. No.: 18/070,636

(22) Filed: Nov. 29, 2022

(51) Int. Cl. G01F 15/02; G01N 9/36; G01N 33/00; G06F 9/54

(52) U.S. Cl. CPC G01F 15/024; G01N 9/36; G01N 33/0073; G06F 9/547

(57) ABSTRACT

A method and system are provided for controlling a flow computer for monitoring a fluid. The method and system involve: establishing communications between the flow computer and a remote computing system through an external application programming interface (API) to obtain from the computing system information of fluid composition of the fluid and/or real-time environmental condition(s); and adjusting a flow measurement operation of the flow computer according to the obtained information of the fluid composition and/or real-time environmental condition(s).

### FIELD

The present disclosure is generally directed to a flow computer, and more particularly, to a method and system of enabling a flow computer to obtain information through an external application programming interface (API) to update a flow computer configuration.

### BACKGROUND

A flow computer can be used in various industries such as the gas and oil industry, chemical manufacturing industries, medical supply industries, and other industries. An industrial process or facility may employ a flow computer to interpret raw sensor data from a flow meter(s) and/or other sensors into readable flow measurement(s) of a fluid, such as liquid or gas, which is being monitored in the process or facility.

### SUMMARY

In accordance with an embodiment, a method and system are provided for controlling a flow computer for monitoring a fluid. The method and system can involve: establishing communications between the flow computer and a remote computing system through an external application programming interface (API) to obtain from the computing system information of a fluid composition of the fluid and/or real-time environmental condition(s); and adjusting a flow measurement operation of the flow computer according to the obtained information of the fluid composition and/or real-time environmental condition(s).

In various embodiments, the method and system can further involve: receiving, in real-time, sensor data from at least one sensor for sensing at least one flow property of the fluid; performing the adjusted flow measurement operation to determine a flow measurement of a flow property of the fluid based on the real-time sensor data; and outputting or storing in real time the determined flow measurement of the property of the fluid. The flow measurement of a flow property can include a measurement of fluid temperature, fluid pressure, fluid density, fluid viscosity, fluid composition, fluid phase, fluid volume, or fluid flow rate.

In various embodiments, in the method and system, the adjusting flow measurement operation can adjust, update, modify or change a flow computer configuration of the flow computer based on the obtained information of the fluid composition and/or real-time environmental condition(s).

In various embodiments, the method and system can further involve: analyzing the information of the fluid composition and/or real-time environmental condition(s), which is relevant to a configuration of the flow measurement operation, to determine whether to adjust the flow measurement operation; and in response to a determination to adjust the flow measurement operation, taking or performing one or more actions to make the adjustments. The method and system also can involve: accessing sensor data from local sensors on the flow computer, wherein the sensor data is analyzed with the information of the fluid composition and/or the real-time environmental condition(s) relevant to the flow measurement operation on the flow computer to determine whether any adjustments to the flow measurement operation are necessary or recommended.

In various embodiments, the application programming interface can comprise an external web-based or cloud-based application programming interface, and the establishing communications operation can comprise: sending a request along with access credentials from the flow computer to the external web-based or cloud-based application programming interface; and in response to the request having valid access credentials, receiving the information of the fluid composition and/or real-time environmental condition(s) through or from the external web-based or cloud-based application programming interface. The communications can be performed across a web-based or cloud-based Supervisory Control and Data Acquisition or SCADA system, which hosts the external web-based or cloud-based application programming interface.

In various embodiments, the flow computer can be arranged in a hazardous area to monitor the fluid in a process or plant, and the fluid can comprise a gas or liquid being monitored by the flow computer.

In various embodiments, the flow computer can be arranged in a medical facility to monitor the fluid in a process or plant, and the fluid can comprise a gas or liquid being monitored by the flow computer.

In various embodiments, the information of the fluid composition can be published to a cloud using the application programming interface or a different application programming interface, by a user using a graphical user interface or web page accessed on a remote computer system, the application programming interface comprising a web-based or cloud-based application programming interface. The information of the fluid composition, which is determined at a laboratory, can be published directly from the remote computer system located at the laboratory to the cloud via the application programming interface or the different application programming interface.

In accordance with an embodiment, a flow computer can include a memory, a communication device and a processor, which is configured to implement various operations described in the above method. In a further embodiment, a system comprises the flow computer, and a cloud which hosts the application programming interface.

In accordance with an embodiment, a tangible computer medium is provided to store computer executable code, which when executed by one or more processors, is configured to implement the above method.

### DESCRIPTION OF THE FIGURES

The description of the various example embodiments is explained in conjunction with the appended drawings.

FIG. 1 illustrates an example computing environment in which a flow computer can access or obtain information for managing, configuring or performing a flow measurement operation from a computing system or device using an application programming interface(s) (API(s)), in accordance with an embodiment.

FIG. 2 is a functional block diagram of example a computing environment in which a flow computer can access or obtain information for managing, configuring or performing a flow measurement operation from a computing system or device using an API(s), such as a web-based or cloud-based API(s), in accordance with an embodiment.

FIG. 3 illustrates an example method by which a flow computer can access or obtain information for managing, configuring or performing a flow measurement operation using an API(s), in accordance with an embodiment.

FIG. 4 illustrates an example method by which a flow computer can access or obtain information for managing, configuring or performing a flow measurement operation using an API(s), in accordance with an embodiment.

FIG. 5 illustrates a block diagram of example components of a computer system (or device), in accordance with an embodiment.

### DISCUSSION OF EXAMPLE EMBODIMENTS

A method and system are provided to enable a flow computer to obtain information, which can be used to configure its flow measurement operation(s), from a remote computing system or device (generally referred to as computing system) through an application programming interface(s) (API(s)), in accordance with various embodiments. The flow computer can be provided with a direct connection to an information system(s) through the use of an API, such as a cloud or web hosted API, to provide, for example, fluid composition information and/or other real-time information (e.g., environmental information such as weather/atmospheric condition(s)) relevant to the configuration and implementation of the flow measurement operation(s) by the flow computer. The fluid being monitored by the flow computer can be a gas and/or liquid flowing through conduits, pipes or other equipment of a process, such as for example an industrial process or plant, in a hazardous area subject to industry standards or requirements. Examples of fluids can include, among other things, crude oil, raw natural gases, water, pure gases such as carbon dioxide, oxygen, or etc., depending on the type of process or plant. Natural gases can for example include a mixture of hydrocarbons such as Methane, Propane, Butane, Pentane, Hexane, etc. Each gas can have different characteristics such as viscosity, density, and boiling point that would impact for example how a particular differential pressure across an orifice plate would correspond to a particular mass flow rate. They also can have different heating values, which can relate to the mass of the fluid to the calories of heat that would be produced by combustion of that fluid. The flow computer can use the information obtained through the use of the API to adjust (e.g., adjust, update, modify or change) the flow measurement operation(s), such as for example the flow computer configuration of the flow computer.

By way of example explanation, a flow computer can be used to determine (e.g., determine, interpret, compute, calculate, generate, derive, etc.) readable measurements of one or more fluid properties from sensor data (e.g., raw sensor signals) received from local sensors such as one or more flow meters (or the like) or other sensors for sensing fluid properties of fluid flowing through a conduit (e.g., conduit, pipes, equipment, etc.). The flow computer can perform flow measurement operation(s) using simple or complex algorithm(s) (or processes), which can involve the use of mathematical expressions (equations) and/or conversion tables, to interpret the input data/signals from the local sensor(s) into readable flow measurement outputs. The flow measurement operation(s), such as the flow computer configuration, can be adjusted according to a material composition of the fluid being monitored and other relevant environmental conditions (or factors). The environmental conditions can include but is not limited to atmospheric pressure, atmospheric or ambient temperature, or other weather/atmospheric conditions or other environmental conditions, which may impact the flow measurements of a fluid by a flow computer.

Fluid composition data, such as chemical composition data for a gas or liquid, can be measured in a lab, which is remote from the fluid monitoring site of the flow computer. A report can be created, distributed manually, and then entered into a control/monitoring system (e.g., a Supervisory Control and Data Acquisition or SCADA system) for controlling and monitoring the process or facility and then communicated to the flow computer which is used to monitor the gas at the monitoring site. This approach involves manual steps, which result in significant delays (e.g., delays for each manual activity to occur). If the lab report information were passed to an API, such as a web-based or cloud-based API, which the flow computer could interrogate, the information would be updated more quickly. This, in turn, would ensure that the flow computer data (similar to money in custody transfer situations) is more up-to-date, and operating more effectively. Other relevant data also may be provided to the flow computer, using an API(s), including real-time environmental condition(s) or other data, which may be relevant to a flow measurement operation.

Accordingly, the method and system, described herein, can provide an improved flow computer, which can interface or directly connect to an API to access or obtain real-time (or updated) data such as fluid composition (e.g., gas or liquid chemical composition) or environmental conditions (e.g., atmospheric pressure, atmospheric or ambient temperature or other weather/atmospheric conditions, etc.) from a data service or API hosted in the cloud or on the web. Such an implementation through an API can significantly speed up the process of retrieving information for use in configuring the flow measurement operation managed and performed by the flow computer, and reduce the need to physically connect (or incorporate) local sensors, e.g., atmospheric pressure sensors, atmospheric temperature sensor or other sensors for sensing other atmospheric properties, to the flow computer.

In some embodiments, the information can be provided to the flow computer through cloud computer from the cloud using the API(s), either automatically such as in real-time or when updated, or upon a request or demand by the flow computer. The information can be requested before each flow run or a plurality of flow runs. The information, such as fluid composition or other information relevant for configuration of the flow measurement operation, can be uploaded, published or pushed to the cloud or a web server (e.g., cloud web server, SCADA-cloud web server, local web server (e.g., managed by a customer), etc.) through the same of different API(s), or uploaded published or pushed to a local server (e.g., a customer server). In some embodiments, the other information relevant to the flow measurement operation can be real-time information such as atmospheric pressure data, atmospheric or ambient temperature, and/or other weather/atmospheric-related information.

In some embodiments, the API(s) can be hosted on a web-based or cloud-based SCADA system for controlling and/or monitoring a process or plant. Direct connectivity can be provided to a user via a web browser/web page to upload, publish or push data to the SCADA cloud. Such a configuration can be particularly practical to implement if the web browser/web page and SCADA system are configured to be on the same private network.

In some embodiments, using a computer system such as a server, a laboratory can employ the cloud or web hosted API to publish the fluid composition information to a cloud or web service which can be accessed by the flow computer with a web API mechanism with the appropriate credentials to extract the information from the cloud or web hosted API, and then automatically apply it to the flow computer.

In some embodiments, at least a portion of the fluid composition information and other real-time information relevant to the flow measurement operation can be provided by one or more users (e.g., lab workers) via a graphical user interface/web-page. The graphical user interface/web-page can allow the users to enter the results of a compositional analysis or upload electronic file containing the compositional analysis.

These and other example features of the method and system of the present disclosure are described below with reference to the Figures.

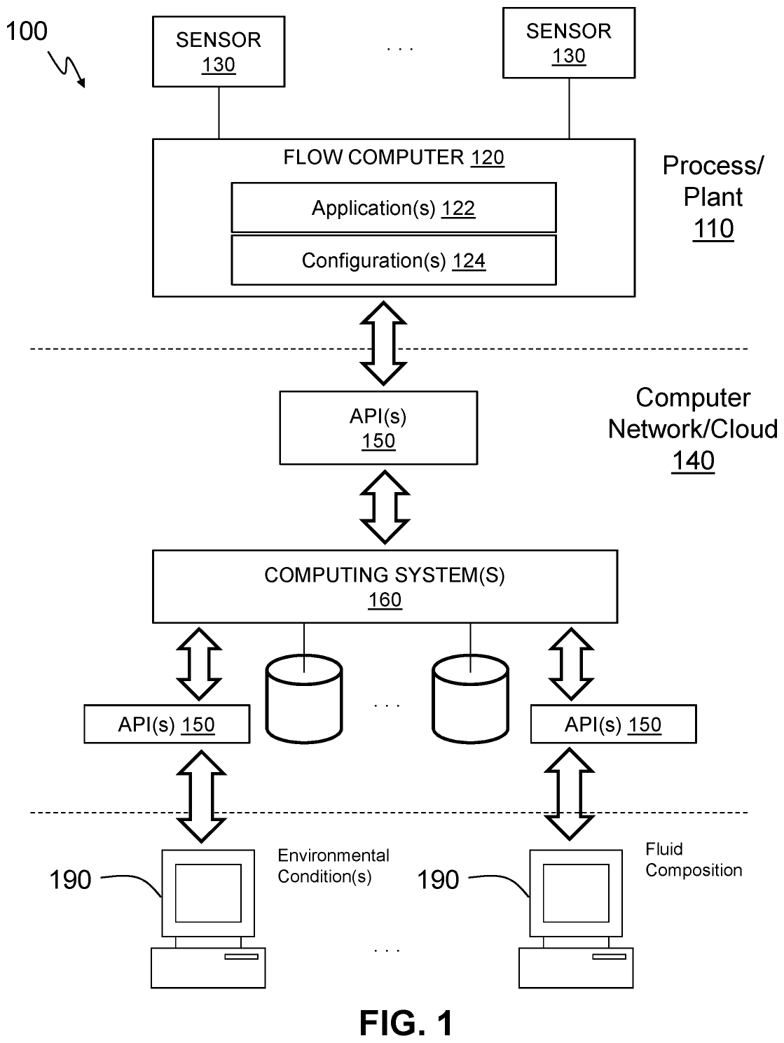

FIG. 1 illustrates an example computing environment 100 in which a flow computer can access or obtain information for managing, configuring or performing a flow measurement operation from a computing system or device using an application programming interface(s) (API(s)), in accordance with an embodiment.

The computing environment 100 can include a flow computer 120, a computing system 160, and application programming interface(s) (API(s)) 150 for enabling the establishment of communications between the flow computer 120 and the computing system to obtain desired information from or through the computing system 160. The flow computer 120 can employ the information received from the computing system 160 to manage, configure and perform its operations, including but not limited to flow measurement operation. In this example, the API(s) 150 can be a web-based or cloud-based API which is hosted on a cloud 140 or computer system or device thereof; however, it is understood that the type of API can be dependent on the application architecture. In various embodiments, the API(s) 150 can be hosted on a computer system or device of a cloud-based SCADA system for monitoring and controlling a process or plant 110. In various embodiments, the API(s) 150 can be hosted on an edge device or gateway or accessed through such devices on a computer network.

The flow computer 120 can be a computer system or device, which is connected to a plurality of sensors 130, e.g., local sensors, for sensing one or more properties of a fluid(s) to be monitored. The sensors 130 can include but is not limited a flow meter(s) or other flow sensor(s), temperature sensor(s), pressure sensor(s) and so forth. The flow meter(s) can include, for example, Coriolis meter, Differential Pressure (DP) meter, Magnetic meter, Multiphase meter, Ultrasonic meter, Vortex meter and/or other flow meter. The flow computer 120 can be configured to implement a flow measurement operation(s), via application(s) (or program(s)) 122 using flow computer configuration(s) 124, to determine (e.g., determine, interpret, calculate, compute, derive, etc.) flow measurement(s) from real-time sensor data (e.g., raw sensor data or signals) from the sensor(s) 130. The flow measurement operation may be implemented according to algorithms (or processes), which can employ mathematical expression(s), to interpret the sensor data to provide for more accurate or precise flow measurement(s) according to fluid properties, sensor properties and other factors which may be relevant to a measurement(s) of a flow property of a fluid. The flow measurement(s) can include, but is not limited to, volume flow or flow rate/speed, heat flow or temperature, mass flow, flow density, fluid state, composition, viscosity, or other measurement of a flow property of a fluid.

The flow computer 120 also can be configured to perform other functions and operations, such as for example: data logging/recording, data (or information) communications, remote metering, providing alarm(s) when the flow measurements do not satisfy predefined threshold(s) or condition(s) or when the sensor(s) or flow computer or its components are not operating within normal tolerances, and performance of other functions and operations. For example, the flow computer 120 can store or record various data such as the flow measurement(s) along with a time stamp in a local or remote memory for review and analysis or reporting. The flow computer 120 also can store data such as events and alarms (including associated time stamp) related to operation of the flow meter and flow measurement(s) for review and analysis or reporting. The flow computer 120 also can output the flow measurement(s) and other data to a monitoring/controlling system (e.g., a cloud-based SCADA system or other similar monitoring/control system) for use in monitoring the fluid and controlling the process or plant 110.

In various embodiments, the flow computer 120 can be employed in a hazardous area in the process or plant 110, and can operate according to IS (intrinsic or intrinsically safe) guidelines or other safety guidelines.

The computing system 160 can be a computer system or device, which can be part of the cloud 140 (or computer network). In this example, the computing system 160 can be a server, such as a web server or data server, which can provide automatically or on-demand/request information (or data) to a remote device, such as the flow computer 120 or other computer device, via API(s) 150. In various embodiments, the information can include fluid composition of fluid being monitored by the flow computer 120, real-time environmental conditions such as atmospheric pressure, ambient or atmospheric temperature or other weather/atmospheric condition, or other information which may be relevant to determination, interpretation, calculation, derivation or computation of flow measurement of a fluid. In various embodiment, the monitoring/controlling system such as a cloud-based SCADA system can be employed to facilitate monitoring, analyzing, reporting and dissemination of data (including information/data discussed herein) to various computer systems and devices.

As further shown in FIG. 1, the computing environment 100 also may include one or more computer systems 190 from which information may be uploaded or published to the cloud 140 (or computer network) for access by the flow computer 120 or other computer systems or devices (e.g., user computer devices such as smartphones, laptops, computer tablets, computers or work stations in the control room, etc.) through an API(s). Each computer system 190 can be configured to provide a graphical user interface (GUI) or access to a web page through which a user can input information (e.g., manually inputting data, or uploading electronic data, designating a source of information/data, etc.) and manage and control the upload or publication of information to the cloud 140 or components thereof. The information may be uploaded manually by a user or automatically uploaded in real-time or upon a change in the data (e.g., change in the fluid composition, environmental condition(s), etc.).

In various embodiments, one computer system 190 can be located in a laboratory where a chemical composition of a fluid can be determined or derived, such as through chromatography or other techniques. The fluid composition results can be uploaded or published to the cloud 140 from the computer system 190 through the API(s) 150, via a network (e.g., Internet, etc.).

Furthermore, another of the computer system 190 can obtain, collect or manage information relating to environmental conditions from one or more sensors and upload or publish them to the cloud 150. The environmental conditions can be uploaded or published in real time through the API(s) 150, and can include atmospheric pressure, atmospheric or ambient temperature, humidity levels, or other weather/atmospheric conditions, which may impact flow measurement of a fluid in the process or plant 110. In various embodiments, the computer system 190 can include a web server to provide real-time weather reporting service.

The API(s) 150 may include a plurality of different APIs. In various embodiments, the same or different API(s) may be used to deliver information from the cloud or to upload or publish information to the cloud. The various systems and devices of the computing environment 100 also may be communicatively connected across a plurality of networks.

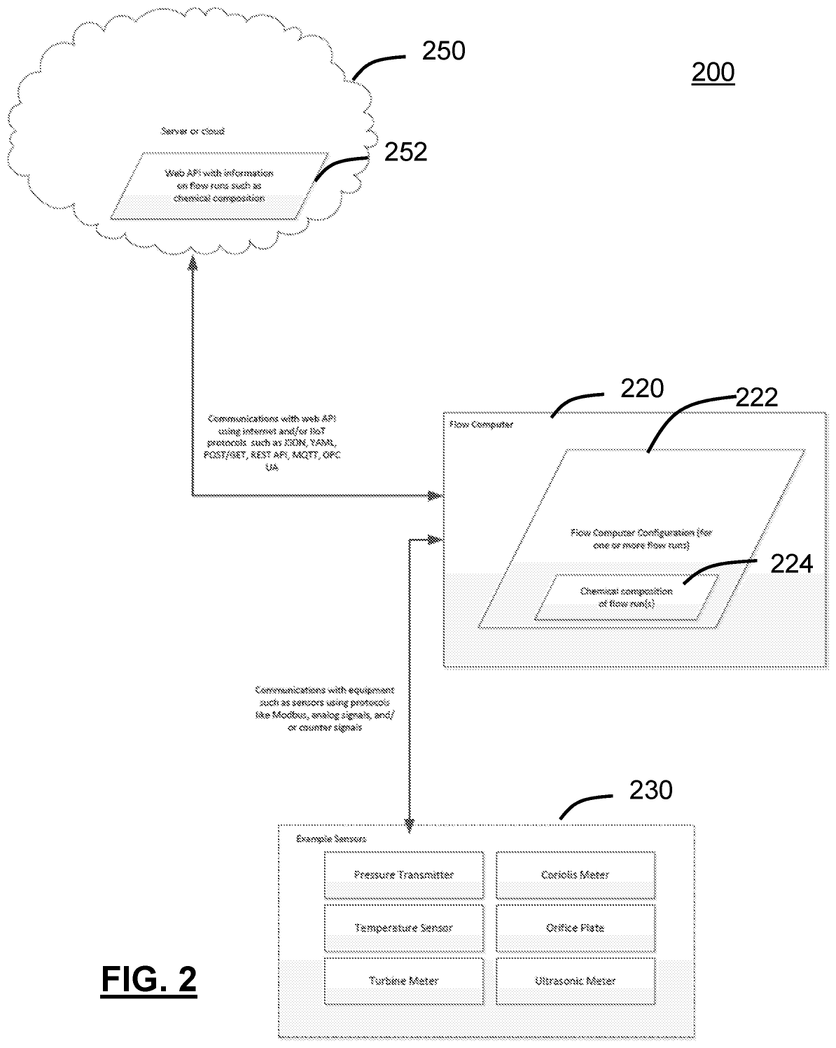

FIG. 2 is a functional block diagram of an example computing environment 200 in which a flow computer 220 can access or obtain information for managing, configuring or performing a flow measurement operation(s), in accordance with an embodiment. In this example, the flow computer 220 is communicatively connected to the computer network 250 or its components which host a web API and to one or more sensors 230. The computer network 250 can be a cloud or cloud network. The sensor(s) 230 can be used to sense one or more flow properties of a fluid in one or more flow runs and output sensor data accordingly (e.g., raw sensor signals). The sensor(s) 230 can include, for example, a pressure transmitter, temperature sensor, turbine meter, Coriolis meter, orifice plate, ultrasonic meter and sensors for sensing other flow properties of fluid in a flow run.

As shown in FIG. 2, the flow computer 220 can perform flow measurement operation(s) to determine flow measurement(s) by determining, interpreting, calculating, computing or deriving flow measurement(s) from real-time sensor data of the sensor(s) 230 according to flow computer configuration 222 (for one or more flow runs) or other factors. The flow computer 220 can perform flow measurement operation(s) using simple or complex algorithms, which can employ mathematical expressions from which to determine, interpret, calculate, compute of derive flow measurement(s). The flow computer configuration 222 can include, or take into account, fluid composition (e.g., chemical composition) 224 of the flow run(s) and other factors such as, for example, environmental condition(s).

The flow computer 220 can communicate with a web API hosted on a computer network 250, such as a networked computer system (e.g., a server) or the cloud (e.g., a plurality of computer systems/servers operating as a single ecosystem). The web API can act as an intermediary to facilitate communications of information between a remote computing system of the cloud and the flow computer 220. In this example, the web API can facilitate communication of information on flow runs such as fluid composition (e.g., chemical composition of the fluid) for the flow runs, as shown in block 252. The flow computer 220 can interface and conduct communication with the web API using Internet and/or IoT (Internet of Things) protocols, such as for example JSON (JavaScript Object Notation), YAML, HTTP (Hypertext Transfer Protocol) requests such as POST/GET, REST API, MQTT (MQ Telemetry Transport), or OPC UA (Open Platform Communications United Architecture).

The flow computer 220 can interface and conduct communication with equipment such as the sensor(s) 230 to receive sensor data from the sensor(s) 230, such as using, for example, protocols like Modbus, analog signals and/or counter signals.

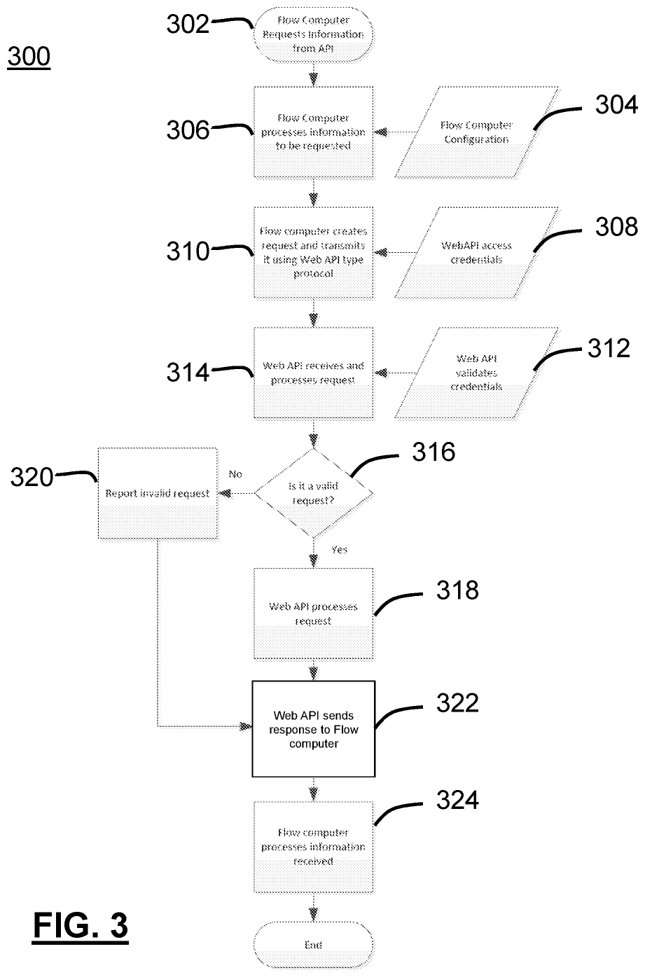

FIG. 3 illustrates an example method 300 by which a flow computer or component(s) can access or obtain information, via at least an API(s), for use in managing, configuring or performing a flow measurement operation using an API(s), in accordance with an embodiment. The method 300 will be described with reference to at least a flow computer, an API and a computing system or components thereof, examples of which are shown and described in FIGS. 1, 2 and 5. In this example, the flow computer can include a processor for managing, configuring or performing a flow measurement operation(s).

The method 300 begins at block 302 in which the flow computer initiates the process for requesting information from the API. As previously discussed, the API can be a web-based or cloud-based API, which can act as an intermediary between application or program running on the flow computer and an information service or system, such as the computing system. The API can be hosted on the cloud, or a cloud-based system such as cloud-based SCADA system for monitoring and controlling a process or plant or equipment associated therewith.

At blocks 304 and 306, the flow computer accesses and evaluates the flow computer configuration, and processes information to be requested through the API. The information to be requested can, for example, be fluid composition for one or more flow runs in a process or plant, and/or other information relevant to flow computer configuration or flow measurement.

At blocks 308 and 310, the flow computer accesses the API access credentials (e.g., WebAPI access credential), creates a request for the information, and transmits the request using a suitable protocol (e.g., Web API type protocol) to the API.

At blocks 312 and 314, the API receives and processes the request, and validates the credentials of the request (or requestor).

At block 316, the API determines whether the request is valid based on the access credentials provided along with the request by the flow computer. If the request is determined to be valid, the API processes the request at block 318. For example, the API can establish communications between the application or program of the flow computer and the computing system to enable transmission of information from the computing system to the flow computer. As previously discussed, this information can include fluid composition, environmental condition(s) and so forth, which can be provided on demand or automatically thereafter in real-time. At block 322, the API sends to the flow computer a response in reply to the request, such as the requested information. At block 324, the flow computer processes the information, which is received through (or from) the API. For example, the flow computer can adjust the flow measurement operation by adjusting, updating, modifying or changing the flow computer configuration with or according to the received information (e.g., fluid composition, environmental condition(s), etc.).

Turning back to block 316, if the request is determined to be invalid, the API reports the request as being invalid at block 320. At block 322, the API sends the response, e.g., invalid request, to the flow computer. At block 324, the flow computer processes the received information, in this case, a report of an invalid request, and can take further action(s), such as re-sending the request with updated or corrected access credential, reporting the invalid request such as to a user, reporting a problem with accessing the requested information, or performing other actions.

The method 300 is provided as an example. In various embodiments, the flow computer can access or obtain information, through the API(s), for use in updating, modifying or changing the flow computer configuration for performing a flow measurement operation(s). It should be understood that the flow computer also can access or obtain other information/data, such as described herein, through the API(s) for managing, configuring or performing other operations of the flow computer (including those described herein), or upload or publish information to the computing system or cloud through the API(s). Furthermore, the information can be obtained through the API(s) before, during or after a flow run, and the flow computer configuration can be adjusted before, during or after a flow run.

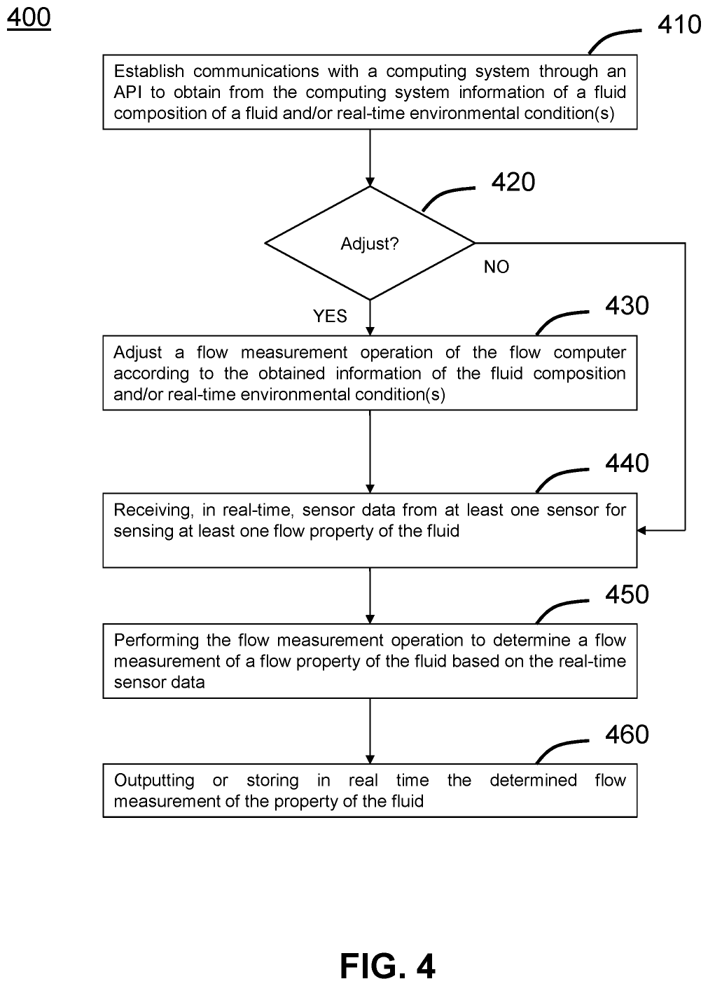

FIG. 4 illustrates an example method 400 by which a flow computer or component(s) can access or obtain information, via at least an API(s), for use in managing, configuring or performing a flow measurement operation using an API(s), in accordance with an embodiment. The method 400 will be described with reference to at least a flow computer, an API and a computing system or components thereof, examples of which are shown and described in FIGS. 1, 2 and 5. In this example, the flow computer can include a processor for managing, configuring or performing a flow measurement operation(s).

The method 400 begins at block 410 in which the flow computer establishes communications with the computing system through an API(s) to obtain from the computing system composition information of fluid to be monitored by the flow computer and/or real-time environmental condition(s) impacting the fluid. For example, the flow computer can request and obtain, either on-demand or automatically, through or from the API(s) information from the computing system or cloud, such as the fluid composition and/or real-time environmental condition(s) or other information relevant to flow computer configuration or flow measurement.

At block 410, the flow computer determines whether to adjust (e.g., adjust, update, modify or change) the flow measurement operation(s) or configuration thereof. For example, the flow computer can determine whether to make such an adjustment using the obtained information if recommended or necessary according to predefined rules or guidelines. In various embodiments, the flow computer can access sensor data from local sensor devices, and analyze the sensor data with the fluid composition information and/or other real-time information relevant to the flow measurement operation(s) to determine if any adjustments to the flow measurement operation(s) are necessary or recommended.

If a determination is made to adjust the flow measurement operation(s), then, at block 430, the flow computer adjusts the flow measurement operation(s) of the flow computer according to the obtained information of the fluid composition and/or real-time environmental condition(s). For example, the flow computer can adjust the flow measurement operation(s) by adjusting the flow computer configuration with or according to the obtained information. If a determination is made not to adjust the flow measurement operation, then the flow computer does not adjust the flow measurement operation. In either case, the method 400 proceeds to block 440 in which the flow computer receives (or continues to receive), in real-time, sensor data from at least one sensor for sensing at least one flow property of the fluid.

At block 450, the flow computer performs the flow measurement operation to determine a flow measurement of a flow property of the fluid based on the real-time sensor data.

At block 460, the flow computer outputs or stores in real-time the determined flow measurement of the property of the fluid. The flow measurement may be stored or outputted along with a time stamp, which can indicate date and time of the measurement event.

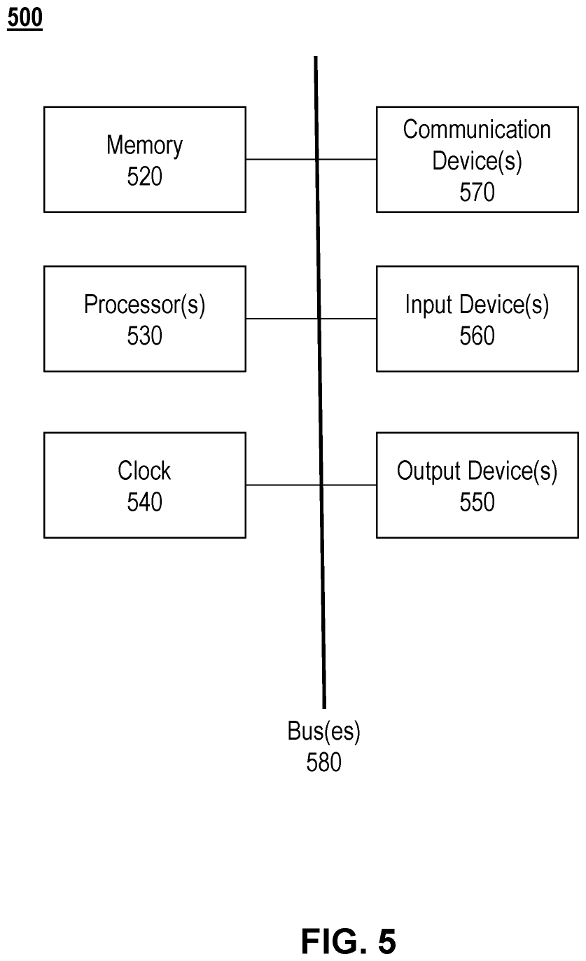

FIG. 5 is a block diagram of example components of a computer system (or device) 500, in accordance with an exemplary embodiment of the present disclosure. As shown in FIG. 5, a computer system 500 can include for example memory 520, processor(s) 530, clock 540, output device 550, input device 560, communication device 570, and a bus system 580 between the components of the computer device. The clock 540 can be used to time-stamp data or an event with a time value (e.g., calendar date/time), and synchronize operations within the device and with remote devices or a system. The communication device 570 can include transmitters and receivers for conducting wireless communications or wireline communications across, for example, electrical wires (e.g., conductors) and/or fiber optic cables.

The memory 520 can store computer executable code, programs, software or instructions, which when executed by a processor(s), controls the operations of the computer device 500, including the various processes described herein. The memory 520 can also store other data used by the computer system 500 or components thereof to perform the operations described herein. The other data can include but is not limited to flow measurement and time stamp, flow computer configuration, sensor data, parameters or thresholds or conditions or rules; alarm or event data, and access credential(s); and other data described herein.

The output device(s) 550 can include a display device, printing device, speaker, lights (e.g., LEDs) and so forth. For example, the output device(s) 550 may output for display or present information regarding the process under control and information associated therewith including process data, alarm occurrences, graphical user interfaces (GUIs), web page(s) or other data.

The input device(s) 560 can include any user input device such as a mouse, trackball, microphone, touch screen, a joystick, control console, keyboard/pad, or other device operable by a user.

The processor(s) 530, which interacts with the other components of the computer system 500, is configured to control or implement the various operations and functions described herein.

The above describes example components of a computer system (or device). The computer system may or may not include all of the components of FIG. 5, and may include other additional components to facilitate operation of the processes and features described herein. The computer system may be a distributed processing system, which includes a plurality of computer systems or devices which can operate to perform the various processes and features described herein.

It should also be understood that the example embodiments disclosed and taught herein are susceptible to numerous and various modifications and alternative forms. Thus, the use of a singular term, such as, but not limited to, “a” and the like, is not intended as limiting of the number of items. Furthermore, the naming conventions for the various components, functions, characteristics, thresholds, and other elements used herein are provided as examples, and can be given a different name or label. The use of the term “or” is not limited to exclusive “or”, but can also mean “and/or”.

It will be appreciated that the development of an actual, real commercial application incorporating aspects of the disclosed embodiments will require many implementation specific decisions to achieve the developer's ultimate goal for the commercial embodiment. Such implementation specific decisions may include, and likely are not limited to, compliance with system related, business related, government related and other constraints, which may vary by specific implementation, location and from time to time. While a developer's efforts might be complex and time consuming in an absolute sense, such efforts would nevertheless be a routine undertaking for those of skill in this art having the benefit of this disclosure.

A processor(s) or controller(s) as described herein can be a processing system, which can include one or more processors, such as CPU, controller, or other processing unit or circuitry, which controls, processes signals or performs the operations of the devices or systems, described herein. Memory/storage devices can include, but are not limited to, disks, solid state drives, optical disks, removable memory devices such as smart cards, SIMs, WIMs, semiconductor memories such as RAM, ROM, PROMS, etc. Transmitting mediums or networks include, but are not limited to, transmission via wireline communication, wireless communication (e.g., Radio Frequency (RF) communication, Bluetooth®, Wi-Fi, Li-Fi, etc.), the Internet, intranets, telephone/modem-based network communication, hard-wired/cabled communication network, satellite communication, and other stationary or mobile network systems/communication links.

In the preceding, reference is made to various embodiments. However, the scope of the present disclosure is not limited to the specific described embodiments. Instead, any combination of the described features and elements, whether related to different embodiments or not, is contemplated to implement and practice contemplated embodiments. Furthermore, although embodiments may achieve advantages over other possible solutions or over the prior art, whether or not a particular advantage is achieved by a given embodiment is not limiting of the scope of the present disclosure. Thus, the preceding aspects, features, embodiments and advantages are merely illustrative and are not considered elements or limitations of the appended claims except where explicitly recited in a claim(s).

The various embodiments disclosed herein may be implemented as a system, method or computer program product. Accordingly, aspects may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects may take the form of a computer program product embodied in one or more computer-readable medium(s) having computer-readable program code embodied thereon.

Any combination of one or more computer-readable medium(s) may be utilized. The computer-readable medium may be a non-transitory computer-readable medium. A non-transitory computer-readable medium may be, for example, but not limited to, an electronic, magnetic, optical, electromagnetic, infrared, or semiconductor system, apparatus, or device, or any suitable combination of the foregoing. More specific examples (a non-exhaustive list) of the non-transitory computer-readable medium can include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. Program code embodied on a computer-readable medium may be transmitted using any appropriate medium, including but not limited to wireless, wireline, optical fiber cable, RF, etc., or any suitable combination of the foregoing.

Computer program code for carrying out operations for aspects of the present disclosure may be written in any combination of one or more programming languages. Moreover, such computer program code can execute using a single computer system or by multiple computer systems communicating with one another (e.g., using a local area network (LAN), wide area network (WAN), the Internet, etc.). While various features in the preceding are described with reference to flowchart illustrations and/or block diagrams, a person of ordinary skill in the art will understand that each block of the flowchart illustrations and/or block diagrams, as well as combinations of blocks in the flowchart illustrations and/or block diagrams, can be implemented by computer logic (e.g., computer program instructions, hardware logic, a combination of the two, etc.). Generally, computer program instructions may be provided to a processor(s) of a general-purpose computer, special-purpose computer, or other programmable data processing apparatus. Moreover, the execution of such computer program instructions using the processor(s) produces a machine that can carry out a function(s) or act(s) specified in the flowchart and/or block diagram block or blocks.

The flowchart and block diagrams in the Figures illustrate the architecture, functionality and/or operation of possible implementations of various embodiments of the present disclosure. In this regard, each block in the flowchart or block diagrams may represent a module, segment or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

While particular embodiments and applications of the present disclosure have been illustrated and described, it is to be understood that the present disclosure is not limited to the precise construction and compositions disclosed herein and that various modifications, changes, and variations can be apparent from the foregoing descriptions without departing from the invention as defined in the appended claims.

1. A method of controlling a flow computer for monitoring a fluid, comprising:
establishing communications between the flow computer and a remote computing system through an external application programming interface (API) to obtain from the computing system information of a fluid composition of the fluid and/or real-time environmental condition(s); and
adjusting a flow measurement operation of the flow computer according to the obtained information of the fluid composition and/or real-time environmental condition(s).

2. The method according to claim 1, further comprising:
receiving, in real-time, sensor data from at least one sensor for sensing at least one flow property of the fluid;
performing the adjusted flow measurement operation to determine a flow measurement of a flow property of the fluid based on the real-time sensor data; and
outputting or storing in real time the determined flow measurement of the property of the fluid.

3. The method according to claim 2, wherein the flow measurement of a flow property comprises a measurement of fluid temperature, fluid pressure, fluid density, fluid viscosity, fluid volume, fluid composition, fluid phase, or fluid flow rate.

4. The method according to claim 1, wherein the adjusting flow measurement operation adjusts, updates, modifies or changes a flow computer configuration of the flow computer based on the obtained information of the fluid composition and/or real-time environmental condition(s).

5. The method according to claim 1, further comprising:
analyzing the information of the fluid composition and/or real-time environmental condition(s), which is relevant to a configuration of the flow measurement operation, to determine whether to adjust the flow measurement operation; and
in response to a determination to adjust the flow measurement operation, taking or performing one or more actions to make the adjustments.

6. The method of claim 5, further comprising:
accessing sensor data from local sensors on the flow computer,
wherein the sensor data is analyzed with the information of the fluid composition and/or the real-time environmental condition(s) relevant to the flow measurement operation on the flow computer to determine whether any adjustments to the flow measurement operation are necessary or recommended.

7. The method according to claim 1, wherein the application programming interface comprises an external web-based or cloud-based application programming interface, the establishing communications comprising:
sending a request along with access credentials from the flow computer to the external web-based or cloud-based application programming interface; and
in response to the request having valid access credentials, receiving the information of the fluid composition and/or real-time environmental condition(s) through or from the external web-based or cloud-based application programming interface.

8. The method according to claim 7, wherein the communications is performed across a web-based or cloud-based Supervisory Control and Data Acquisition or SCADA system, which hosts the external web-based or cloud-based application programming interface.

9. The method according to claim 1, wherein the flow computer is arranged in a hazardous area to monitor the fluid in a process or plant, and the fluid comprises a gas or liquid being monitored by the flow computer.

10. The method according to claim 1, wherein the flow computer is arranged in a facility to monitor the fluid in a process or plant, and the fluid comprises a gas or liquid being monitored by the flow computer.

11. The method according to claim 1, wherein the information of the fluid composition is published to a cloud using the application programming interface or a different application programming interface, by a user using a graphical user interface or web page accessed on a remote computer system, the application programming interface comprising a web-based or cloud-based application programming interface.

12. The method of claim 11, wherein the information of the fluid composition, which is determined at a laboratory, is published directly from the remote computer system located at the laboratory to the cloud via the application programming interface or the different application programming interface.

13. A flow computer comprising:
a memory;
a communication device; and
a processor configured to:
establish communications between the flow computer and a remote computing system through an external application programming interface (API) to obtain from the computing system information of a fluid composition of the fluid and/or real-time environmental condition(s); and
adjust a flow measurement operation of the flow computer according to the obtained information of the fluid composition and/or real-time environmental condition(s).

14. The flow computer according to claim 13, wherein the process is further configured to:
receive, in real-time, sensor data from at least one sensor for sensing at least one flow property of the fluid;
perform the adjusted flow measurement operation to determine a flow measurement of a flow property of the fluid based on the real-time sensor data; and
output or store in real time the determined flow measurement of the property of the fluid.

15. The flow computer according to claim 14, wherein the flow measurement of a flow property comprises a measurement of fluid temperature, fluid pressure, fluid density, fluid viscosity, fluid composition, fluid phase, fluid volume, or fluid flow rate.

16. The flow computer according to claim 13, wherein, to adjust flow measurement operation, the processor is configured to adjust, update, modify or change a flow computer configuration of the flow computer based on the obtained information of the fluid composition and/or real-time environmental condition(s).

17. The flow computer according to claim 13, wherein the processor is further configured to:
analyze the information of the fluid composition and/or real-time environmental condition(s), which is relevant to a configuration of the flow measurement operation, to determine whether to adjust the flow measurement operation; and
in response to a determination to adjust the flow measurement operation, take or perform one or more actions to make the adjustments.

18. The flow computer of claim 17, wherein the processor is further configured to:
access sensor data from local sensors on the flow computer,
wherein the sensor data is analyzed by the processor with the information of the fluid composition and/or the real-time environmental condition(s) relevant to the flow measurement operation on the flow computer to determine whether any adjustments to the flow measurement operation are necessary or recommended.

19. The flow computer according to claim 13, wherein the application programming interface comprises an external web-based or cloud-based application programming interface,
to establish communications, the processor is configured to:
send a request along with access credentials from the flow computer via the communication device to the external web-based or cloud-based application programming interface; and
in response to the request having valid access credentials, receive via the communication device the information of the fluid composition and/or real-time environmental condition(s) through or from the external web-based or cloud-based application programming interface.

20. The flow computer according to claim 19, wherein the communications is performed across a web-based or cloud-based Supervisory Control and Data Acquisition or SCADA system, which hosts the external web-based or cloud-based application programming interface.

21. The flow computer according to claim 13, wherein the flow computer is arranged in a hazardous area to monitor the fluid in a process or plant, and the fluid comprises a gas or liquid being monitored by the flow computer.

22. The flow computer according to claim 13, wherein the flow computer is arranged in a facility to monitor the fluid in a process or plant, and the fluid comprises a gas or liquid being monitored by the flow computer.

23. The flow computer according to claim 13, wherein the information of the fluid composition is published to a cloud using the application programming interface or a different application programming interface, by a user using a graphical user interface or web page accessed on a remote computer system, the application programming interface comprising a web-based or cloud-based application programming interface.

24. The flow computer of claim 23, wherein the information of the fluid composition, which is determined at a laboratory, is published directly from the remote computer system located at the laboratory to the cloud via the application programming interface or the different application programming interface.

25. A system comprising:
the flow computer according to claim 13;
a cloud which hosts the application programming interface, which comprises a web-based or cloud-based application programming interface.

26. A tangible computer medium storing computer executable code, which when executed by one or more processors, is configured to implement a method of controlling a flow computer for monitoring a fluid, the method comprising:
establishing communications between the flow computer and a remote computing system through an external application programming interface (API) to obtain from the computing system information of a fluid composition of the fluid and/or real-time environmental condition(s); and
adjusting a flow measurement operation of the flow computer according to the obtained information of the fluid composition and/or real-time environmental condition(s).
